SEQUENCE LISTING (72 sequences)

SEQ ID NO: 1 - DNA, 15 bp, Artificial Sequence
  Aptamer (no stabilization domain)
caatgtacagtattg

SEQ ID NO: 2 - DNA, 31 bp, Artificial Sequence
  Aptamer (no stabilization domain) elongated duplex
aacagttgcgcagcctcaatgtacagtattg

SEQ ID NO: 3 - DNA, 35 bp, Artificial Sequence
  Aptamer (4 bp stabilization domain)
gttgcgcagcctacgccaatgtacagtattggcgt

SEQ ID NO: 4 - DNA, 51 bp, Artificial Sequence
  Aptamer (12 bp stabilization domain)
gttgcgcagcctcgcagaggtgagcaatgtacagtattgctcacctctgcg

SEQ ID NO: 5 - DNA, 18 bp, Artificial Sequence
  18 bp Target Specific Strand
aggctgcgcaactgttgg

SEQ ID NO: 6 - DNA, 40 bp, Artificial Sequence
  40 bp Target Specific Strand
aggctgcgcaactgttgggaagggcgatcggtgcgggcct

SEQ ID NO: 7 - DNA, 100 bp, Artificial Sequence
  100 bp Target Specific Strand
aggctgcgcaactgttgggaagggcgatcggtgcgggcctcttcgctattacgccagctggcgaaagggggatgtgctgcaaggcgattaagttgggtaa

SEQ ID NO: 8 - DNA, 18 bp, Artificial Sequence
  18 bp Target
ccaacagttgcgcagcct

SEQ ID NO: 9 - DNA, 40 bp, Artificial Sequence
  40 bp Target
aggcccgcaccgatcgcccttcccaacagttgcgcagcct

SEQ ID NO: 10 - DNA, 100 bp, Artificial Sequence
  100 bp Target
ttacccaacttaatcgccttgcagcacatccccctttcgccagctggcgtaatagcgaagaggcccgcaccgatcgcccttcccaacagttgcgcagcct

SEQ ID NO: 11 - DNA, 15 bp, Artificial Sequence
  Aptamer (no stabilization domain) (0bp duplex with target specific strand)
caatgtacagtattg

SEQ ID NO: 12 - DNA, 21 bp, Artificial Sequence
  Aptamer (no stabilization domain) (6bp duplex with target specific strand)
cagcctcaatgtacagtattg

SEQ ID NO: 13 - DNA, 27 bp, Artificial Sequence
  Aptamer (no stabilization domain) (12bp duplex with target specific strand)
gttgcgcagcctcaatgtacagtattg

SEQ ID NO: 14 - DNA, 33 bp, Artificial Sequence
  Aptamer (no stabilization domain) (18bp duplex with target specific strand)
ccaacagttgcgcagcctcaatgtacagtattg

SEQ ID NO: 15 - DNA, 39 bp, Artificial Sequence
  Aptamer (no stabilization domain) (24bp duplex with target specific strand)
cccttcccaacagttgcgcagcctcaatgtacagtattg

SEQ ID NO: 16 - DNA, 45 bp, Artificial Sequence
  Aptamer (no stabilization domain) (30bp duplex with target specific strand)
cgatcgcccttcccaacagttgcgcagcctcaatgtacagtattg

SEQ ID NO: 17 - DNA, 27 bp, Artificial Sequence
  Aptamer (6bp stabilization domain) (0 bp duplex with target specific strand)
actggccaatgtacagtattggccagt

SEQ ID NO: 18 - DNA, 33 bp, Artificial Sequence
  Aptamer (6bp stabilization domain) (6 bp duplex with target specific strand)
cagcctactggccaatgtacagtattggccagt

SEQ ID NO: 19 - DNA, 39 bp, Artificial Sequence
  Aptamer (6bp stabilization domain) (12bp duplex with target specific strand)
gttgcgcagcctactggccaatgtacagtattggccagt

SEQ ID NO: 20 - DNA, 45 bp, Artificial Sequence
  Aptamer (6bp stabilization domain) (18bp duplex with target specific strand)
ccaacagttgcgcagcctactggccaatgtacagtattggccagt

SEQ ID NO: 21 - DNA, 51 bp, Artificial Sequence
  Aptamer (6bp stabilization domain) (24bp duplex with target specific strand)
cccttcccaacagttgcgcagcctactggccaatgtacagtattggccagt

SEQ ID NO: 22 - DNA, 57 bp, Artificial Sequence
  Aptamer (6bp stabilization domain) (30bp duplex with target specific strand)
cgatcgcccttcccaacagttgcgcagcctactggccaatgtacagtattggccagt

SEQ ID NO: 23 - DNA, 40 bp, Artificial Sequence; identical to SEQ ID NO: 6
  Target Specific Strand

SEQ ID NO: 24 - DNA, 34 bp, Artificial Sequence
  Low Tm Aptamer
gttgcgcagcctttatcaatgtacagtatgataa

SEQ ID NO: 25 - DNA, 35 bp, Artificial Sequence
  Medium Tm Aptamer
gttgcgcagcctcgaccaatgtacagtattggtcg

SEQ ID NO: 26 - DNA, 43 bp, Artificial Sequence
  High Tm Aptamer
gttgcgcagcctgacacgaccaatgtacagtattggtcgtgtc

SEQ ID NO: 27 - DNA, 18 bp, Artificial Sequence
  Target Specific Strand
aggctgcgcaactgttgg

SEQ ID NO: 28 - DNA, 33 bp, Artificial Sequence
  miR-21-5p Aptamer
gactgatgttgacgccaatgtacagtattggcg

SEQ ID NO: 29 - DNA, 18 bp, Artificial Sequence
  miR-21 Specific Strand
tcaacatcagtctgataa

SEQ ID NO: 30 - DNA, 33 bp, Artificial Sequence
  miR-223-5p Aptamer
acaagctgagttcgccaatgtacagtattggcg

SEQ ID NO: 31 - DNA, 18 bp, Artificial Sequence
  miR-223 Specific Strand
aactcagcttgtcaaata

SEQ ID NO: 32 - DNA, 35 bp, Artificial Sequence
  miR-1827 Aptamer
agtagattgaatcggacaatgtacagtattgtccg

SEQ ID NO: 33 - DNA, 18 bp, Artificial Sequence
  miR-1827 Specific Strand
attcaatctactgcctca

SEQ ID NO: 34 - DNA, 33 bp, Artificial Sequence
  GAPDH Aptamer
ttctcaagacggaatcaatgtacagtattgatt

SEQ ID NO: 35 - DNA, 18 bp, Artificial Sequence
  GAPDH Specific Strand
ccgtcttgagaaacctgc

SEQ ID NO: 36 - DNA, 33 bp, Artificial Sequence
  ACTB Aptamer
acgcaactaagtgaccaatgtacagtattggtc

SEQ ID NO: 37 - DNA, 18 bp, Artificial Sequence
  ACTB Specific Strand
acttagttgcgttacacc

SEQ ID NO: 38 - DNA, 35 bp, Artificial Sequence
  hnRNPA2B1 Aptamer
tgcctatcagtatgaccaatgtacagtattggtca

SEQ ID NO: 39 - DNA, 18 bp, Artificial Sequence
  hnRNPA2B1 Specific Strand
tactgataggcagtctgg

SEQ ID NO: 40 - DNA, 35 bp, Artificial Sequence
  GAS5 Aptamer
tgtcttcatgtctagccaatgtacagtattggcta

SEQ ID NO: 41 - DNA, 18 bp, Artificial Sequence
  GAS5 Specific Strand
gacatgaagacagttcct

SEQ ID NO: 42 - DNA, 33 bp, Artificial Sequence
  PCGEM1 Aptamer
cctcagaaatctcggcaatgtacagtattgccg

SEQ ID NO: 43 - DNA, 18 bp, Artificial Sequence
  PCEGM1 Specific Strand
agatttctgaggggaatt

SEQ ID NO: 44 - DNA, 33 bp, Artificial Sequence
  NEAT1 Aptamer
ttagcgccaaactagcaatgtacagtattgcta

SEQ ID NO: 45 - DNA, 18 bp, Artificial Sequence
  NEAT1 Specific Strand
gtttggcgctaaactctt

SEQ ID NO: 46 - DNA, 33 bp, Artificial Sequence
  miR-21-5p NOT Aptamer
tcagtctgataacgccaatgtacagtattggcg

SEQ ID NO: 47 - DNA, 33 bp, Artificial Sequence
  miR-223-5p NOT Aptamer
gcttgtcaaatacgccaatgtacagtattggcg

SEQ ID NO: 48 - DNA, 35 bp, Artificial Sequence
  miR-1827 NOT Aptamer
tctactgcctcacggacaatgtacagtattgtccg

SEQ ID NO: 49 - DNA, 33 bp, Artificial Sequence
  GAPDH NOT Aptamer
tgagaaacctgcaatcaatgtacagtattgatt

SEQ ID NO: 50 - DNA, 33 bp, Artificial Sequence
  ACTB NOT Aptamer
ttgcgttacaccgaccaatgtacagtattggtc

SEQ ID NO: 51 - DNA, 35 bp, Artificial Sequence
  hnRNPA2B1 NOT Aptamer
taggcagtctggtgaccaatgtacagtattggtca

SEQ ID NO: 52 - DNA, 35 bp, Artificial Sequence
  GAS5 NOT Aptamer
aagacagttccttagccaatgtacagtattggcta

SEQ ID NO: 53 - DNA, 33 bp, Artificial Sequence
  PCGEM1 NOT Aptamer
ctgaggggaattcggcaatgtacagtattgccg

SEQ ID NO: 54 - DNA, 33 bp, Artificial Sequence
  NEAT1 NOT Aptamer
cgctaaactctttagcaatgtacagtattgcta

SEQ ID NO: 55 - DNA, 33 bp, Artificial Sequence
  Aptamer (short target)
agtagattgaatcgccaatgtacagtattggcg

SEQ ID NO: 56 - DNA, 18 bp, Artificial Sequence
  Short Target Specific Strand (originally A)
attcaatctactgtctca

SEQ ID NO: 57 - DNA, 18 bp, Artificial Sequence
  Short Target Specific Strand (originally C)
attcaatctactggctca

SEQ ID NO: 58 - DNA, 18 bp, Artificial Sequence
  Short Target Specific Strand (originally T)
attcaatctactgactca

SEQ ID NO: 59 - DNA, 18 bp, Artificial Sequence
  Short Target Specific Strand (originally G)
attcaatctactgcctca

SEQ ID NO: 60 - DNA, 18 bp, Artificial Sequence
  Short Target (A)
tgagacagtagattgaat

SEQ ID NO: 61 - DNA, 18 bp, Artificial Sequence
  Short Target (C)
tgagccagtagattgaat

SEQ ID NO: 62 - DNA, 18 bp, Artificial Sequence
  Short Target (T)
tgagtcagtagattgaat

SEQ ID NO: 63 - DNA, 18 bp, Artificial Sequence
  Short Target (G)
tgaggcagtagattgaat

SEQ ID NO: 64 - DNA, 33 bp, Artificial Sequence
  Aptamer (long target)
tgcctatcagtacgccaatgtacagtattggcg

SEQ ID NO: 65 - DNA, 18 bp, Artificial Sequence
  Long Target Specific Strand (originally A)
tactgataggcagtctgt

SEQ ID NO: 66 - DNA, 18 bp, Artificial Sequence
  Long Target Specific Strand (originally C)
tactgataggcagtctgg

SEQ ID NO: 67 - DNA, 18 bp, Artificial Sequence
  Long Target Specific Strand (originally T)
tactgataggcagtctga

SEQ ID NO: 68 - DNA, 18 bp, Artificial Sequence
  Long Target Specific Strand (originally G)
tactgataggcagtctgc

SEQ ID NO: 69 - DNA, 40 bp, Artificial Sequence
  Long Target (A)
cttttctttacagactgcctatcagtaattatctcaatgg

SEQ ID NO: 70 - DNA, 40 bp, Artificial Sequence
  Long Target (C)
cttttctttccagactgcctatcagtaattatctcaatgg

SEQ ID NO: 71 - DNA, 40 bp, Artificial Sequence
  Long Target (T)
cttttcttttcagactgcctatcagtaattatctcaatgg

SEQ ID NO: 72 - DNA, 40 bp, Artificial Sequence
  Long Target (G)
cttttctttgcagactgcctatcagtaattatctcaatgg

(19) United States
(12) Patent Application Publication
(10) Pub. No.: US 2023/0357857 A1
(43) Pub. Date: Nov. 9, 2023

(54) NUCLEIC ACID NANOSTRUCTURES WITH TUNABLE FUNCTIONAL STABILITY

(71) Applicant: National University of Singapore, Singapore (SG)

(72) Inventors: Huilin Shao, Singapore (SG); Yuan Chen, Singapore (SG); Rui Yuan Nicholas Ho, Singapore (SG)

(73) Assignee: National University of Singapore, Singapore (SG)

(21) Appl. No.: 18/028,432

(22) PCT Filed: Sep. 23, 2021
(86) PCT No.: PCT/SG2021/050576

§ 371 Date: Mar. 24, 2023

(30) Foreign Application Priority Data
Sep. 25, 2020 (SG) 10202009491W

(51) Int. Cl. C12Q 1/6886; C12Q 1/682

(52) U.S. Cl. CPC C12Q 1/6886; C12Q 1/682; C12Q 2600/16; C12Q 2600/158

(57) ABSTRACT

The present invention relates to catalytic, nucleic acid nanostmctures that enable versatile detection of RNAs, their use, and devices comprising same. The nanostructure comprises a DNA polymerase enzyme, a DNA aptamer and an inverter oligonucleotide, wherein the DNA aptamer comprising (i) a conserved sequence region for binding to the DNA polymerase enzyme, wherein the binding inactivates the polymerase activity, (ii) a variable sequence region for binding to the inverter oligonucleotide, and (iii) a duplex stabilizer region that lies between the conserved sequence region and the variable sequence region. The present invention also relates to the use of the nanostructure in a method of detection of nucleic acid for diagnosing a disease in a subject.

### FIELD OF THE INVENTION

The present invention relates to catalytic, nucleic acid nanostructures that enable versatile detection of RNAs. More specifically, the nanostructures are formed through the hybrid assembly of nucleic acids and enzymes; upon their specific hybridization to the intended nucleic acid targets (e.g., RNA), the nanostructures are activated, thereby liberating active enzymes. With rational design, the nanostructures developed harbour different functional stability to recognize a wide variety of RNA targets, of different lengths and sequence content.

### BACKGROUND OF THE INVENTION

Circulating RNAs have recently emerged as a promising, minimally-invasive biomarkers to empower new disease diagnostics. Despite such clinical potential, the large diversity of circulating RNAs—different molecular types have different lengths and sequence contents—poses significant challenges to their clinical translation. For the analysis of long RNA targets (e.g., mRNA and IncRNA), commercial assays leverage primarily polymerase chain reaction (PCR) to amplify and detect; such process not only necessitates conversion to DNA, but also requires bulky and specialized operation. For the detection of short RNA targets (e.g., miRNA), the process is notably more challenging. As short RNA targets do not have sufficient span to bind with pairs of amplification primers, as necessary in conventional PCR, dedicated assay design and extensive processing are required to modify, amplify and analyze short RNA targets; the approach thus becomes increasing costly and challenging to multiplex.

There is a need for a molecular platform to enable direct rapid, visual and modular detection of RNA.

### SUMMARY OF THE INVENTION

Instead of relying on target nucleic acid amplification, the technology of the present invention provides catalytic, nucleic acid nanostructures that enable versatile detection of RNAs. The nanostructures are formed through the hybrid assembly of nucleic acids and enzymes; upon their specific hybridization to the intended nucleic acid targets (e.g., RNA), the nanostructures are destabilized and activated, thereby liberating active enzymes. With rational design, the nanostructures are developed to harbor different functional stability to recognize a wide variety of RNA targets (of different lengths and sequence content), including single base mutations. Moreover, the system is compatible with different readout modalities, in different environments, for enhanced portability. Further, the system is capable of multiplexed molecular logic operations in a one-pot reaction. The system is able to:
i) equalize different target concentration,
ii) perform multiplier functions (multiple targets), and
iii) implement thresholding functionality

In a first aspect there is provided a recognition nanostructure comprising a DNA polymerase enzyme-specific DNA aptamer having a conserved sequence region that binds to inactivate DNA polymerase, a variable sequence region comprising a segment that is complementary to a portion of a target-specific inverter oligonucleotide, and a duplex stabilizer region that lies between the conserved aptamer sequence region and the variable sequence region, wherein variation of the length and/or composition of the duplex stabilizer region can vary the conformational stability of the nanostructure.

In some embodiments the variable sequence region is at least 8 nucleotides in length.

In some embodiments the recognition nanostructure comprises a target-specific inverter oligonucleotide, wherein a portion of the target-specific inverter oligonucleotide forms a duplex with the variable sequence region and a portion of the target-specific inverter oligonucleotide forms an overhang of at least 4 nucleotides.

In some embodiments increasing the length and/or the GC content of the duplex stabilizer domain increases the conformational stability of the nanostructure, thereby altering;
i) target compatibility, such as the range of targets; and/or
ii) kinetics of enzyme activation; and/or
iii) ability to measure inputs, such as temperature and chemicals used that affect hydrogen bonding.

In some embodiments the duplex stabilizer domain length is in the range of 0 to 20 nucleic acids, in the range of 0 to 15 nucleic acids, in the range of 3 to 12 nucleic acids, preferably in the range of 3 to 6 nucleic acids.

In some embodiments the duplex stabilizer region comprises or consists of a duplex stabilizer region nucleic acid sequence set forth in any of Tables 1 to 4.

More particularly, the duplex stabilizer region comprises or consists of a nucleic acid sequence selected from the group comprising SEQ ID NO: 3, SEQ ID NO: 4, SEQ ID NO: 17, SEQ ID NO: 18, SEQ ID NO: 19, SEQ ID NO: 20, SEQ ID NO: 21, SEQ ID NO: 22, SEQ ID NO: 24, SEQ ID NO: 25, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53 and SEQ ID NO: 54.

In a second aspect there is provided a method of detecting target nucleic acids in a sample, comprising the steps of:
(a) providing a sample comprising nucleic acid;
(b) providing a composition comprising a DNA polymerase enzyme, a recognition nanostructure defined in any aspect of the invention, and a target-specific inverter oligonucleotide; or
(c) providing a composition comprising a DNA polymerase enzyme and a recognition nanostructure defined in any aspect of the invention;
(d) contacting the sample comprising nucleic acid with the composition of (b) or (c), wherein target nucleic acid binding to the inverter oligonucleotide destabilizes the recognition nanostructure, thereby releasing the DNA polymerase enzyme from inhibition by the DNA aptamer;
(e) detecting DNA polymerase enzyme activity, wherein the intensity of activity indicates the presence of target nucleic acid in the sample.

In a third aspect there is provided a method of diagnosing a disease in a subject, comprising the steps of:
(a) providing a sample comprising nucleic acid;
(b) providing a composition comprising a DNA polymerase enzyme, a recognition nanostructure defined in any aspect of the invention, and a target-specific inverter oligonucleotide; or
(c) providing a composition comprising a DNA polymerase enzyme and a recognition nanostructure defined in any aspect of the invention;
(d) contacting the sample comprising nucleic acid with the composition of (b) or (c), wherein target nucleic acid binding to the inverter oligonucleotide destabilizes the recognition nanostructure, thereby releasing the DNA polymerase enzyme from inhibition by the DNA aptamer;
(e) detecting DNA polymerase enzyme activity, wherein the intensity of activity indicates the presence of target nucleic acid in the sample;
(f) diagnosing the patient with the disease when presence of target nucleic acid in the sample is detected.

In some embodiments of the second or third aspect the inverter oligonucleotide is about 18 to 45 nucleotides in length.

In some embodiments of the first to third aspects the method further comprises providing one or more additional recognition nanostructures complementary to one or more target nucleic acids different from the target nucleic acid of a first recognition nanostructure in the sample, for multiplex detection.

In some embodiments each of the recognition nanostructures comprises a combination of DNA aptamer: inverter oligonucleotide: DNA polymerase enzyme in a ratio to form a logic gate selected from a group comprising AND, OR, NOT, NAND and NOR.

In some embodiments the combination of DNA aptamer: inverter oligonucleotide: DNA polymerase enzyme ratio of each recognition nanostructure is selected from the group (i) to (v) comprising:
(i) two nanostructures each having 1:1:0.5 DNA aptamer:inverter oligonucleotide:DNA polymerase enzyme ratio to form a AND logic gate;
(ii) two nanostructures each having 1:1:1 DNA aptamer:inverter oligonucleotide:DNA polymerase enzyme ratio to form a OR logic gate;
(iii) one nanostructure having 1:0:1 to form a NOT logic gate;
(iv) two nanostructures each having 1:0:1 DNA aptamer:inverter oligonucleotide:DNA polymerase enzyme ratio to form a NAND gate; and
(v) two nanostructures each having 1:0:0.5 DNA aptamer:inverter oligonucleotide:DNA polymerase enzyme ratio to form a NOR gate.

In some embodiments the relative amount of inverter oligonucleotide in the combination of DNA aptamer:inverter oligonucleotide:DNA polymerase enzyme ratio of each recognition nanostructure is adjusted to equalize the DNA polymerase enzyme activity when there are differences in levels of a plurality of targets in the sample.

In some embodiments the combination of DNA nanostructures and the ratio of the DNA aptamer, inverter oligonucleotide, and DNA polymerase in each nanostructure is varied to provide (a) a multi-input OR gate or (b) a multi-input AND gate.

In some embodiments the combination of DNA nanostructures and the ratio of the DNA aptamer, inverter oligonucleotide, and DNA polymerase in each nanostructure is varied to provide a threshold level of detection of a plurality of targets in a sample.

In some embodiments the disease is NSCLC and the recognition nanostructures detect a plurality of RNA species selected from the group comprising miR-21-5p, miR-223-5p, GAPDH, hnRNPA2B1 and GAS5 or combinations thereof.

NSCLC is any type of epithelial lung cancer other than small cell lung cancer (SCLC). The most common types of NSCLC are squamous cell carcinoma, large cell carcinoma, and adenocarcinoma, but there are several other types that occur less frequently, and all types can occur in unusual histologic variants.

In some embodiments the recognition nanostructure oligonucleotide sequence is selected from the sequences in Table 4.

More particularly, one or more recognition nanostructure oligonucleotide sequences are selected from the group comprising SEQ ID NOs: 28, SEQ ID NO: 30, SEQ ID NO: 32, SEQ ID NO:34, SEQ ID NO: 36, SEQ ID NO: 38, SEQ ID NO: 40, SEQ ID NO: 42, SEQ ID NO: 44, SEQ ID NO: 46, SEQ ID NO: 47, SEQ ID NO: 48, SEQ ID NO: 49, SEQ ID NO: 50, SEQ ID NO:51, SEQ ID NO: 52, SEQ ID NO: 53 and SEQ ID NO: 54. Preferably, one or more of recognition nanostructure oligonucleotide sequences are selected from the group comprising SEQ ID NO: 28, SEQ ID NO: 30, SEQ ID NO: 34, SEQ ID NO: 38 and SEQ ID NO: 40.

In a fourth aspect there is provided a device comprising:
(i) composition b) or composition c) comprising at least one DNA polymerase enzyme and at least one recognition nanostructure, as defined in any one of the previous claims, at a 1^{st} location;
(ii) signaling nanostructures comprising a self-priming portion responsive to active DNA polymerase enzyme, as defined in any one of the previous claims, attached at a 2^{nd} location; and
(iii) an intermediate stage for mixing of said detection nanostructures with sample nucleic acid to release active enzyme to said 2^{nd} location.

In some embodiments the device is selected from the group comprising a microfluidic device and a lateral flow device.

In some embodiments the device is a microfluidic device comprising:
(i) a common signaling cartridge configured to receive one or more assay cassettes, wherein the cartridge comprises a base with membranes embedded to immobilize signaling nanostructures, and a common outlet which makes fluid connection with said 2^{nd} location in each of the one or more assay cassettes;
(ii) one or more assay cassettes each comprising, at a 1^{st} location, an inlet and at least one DNA polymerase enzyme and at least one recognition nanostructure; an intermediate stage microchannel in fluid connection between the 1^{st} and 2^{nd} locations, for mixing of said detection nanostructures with sample nucleic acid to release active enzyme to said 2^{nd} location;
   wherein, when the device is assembled and in use, there is fluidic flow from the sample inlet to the common outlet, actuated by a withdrawal septum.

In a fifth aspect there is provided a nucleic acid detection kit comprising;
(a) a composition comprising at least one DNA polymerase enzyme and at least one recognition nanostructure defined in the first aspect;
(b) a signaling nanostructure that is reactive to active DNA polymerase enzyme, wherein the signaling nanostructure comprises a self-priming portion responsive to the DNA polymerase enzyme; or
(c) labelled nucleotides (dNTPs) and signal development reagents, wherein active DNA polymerase enzyme adds labelled nucleotides to the signaling nanostructure and the signal development reagents bind to the labelled nucleotides incorporated into the self-primed portion,
or any combination thereof.

In some embodiments (a) to (c) are as defined according to any aspect of the invention.

According to any aspect of the present invention, at least one of the aptamer and/or inverter and/or signaling nanostructure oligonucleotides are structurally and/or chemically modified from their natural nucleic acid.

In some embodiments, said structural and/or chemical modifications are selected from the group comprising the addition of phosphorothioate (PS) bonds, 2′-O-Methyl modifications, phosphoramidite C3 Spacers and 5′ additions such as amino, thiol, acryldite or azide groups during synthesis.

### BRIEF DESCRIPTION OF FIGURES

FIG. 1 shows the effects of the length of the stabilizer domain. (Top panel)

Schematics on the assembly of different nanostructures, each bearing a different-length stabilizer domain:(a) 3 base pair (bp), (b) 12 bp and (c and d) 0 bp and their assembly into a recognition nanostructure with either (a, b and c) a 12-bp or (d) a 16-bp duplexed variable region. Not drawn to scale. (Bottom panel) Resultant polymerase activity, after nanostructure incubation with different samples. In the absence of target, minimal signal was observed across all nanostructures. In the presence of targets (of different lengths; 18 bases, 40 bases and 100 bases), only the nanostructure with the 3-bp stabilizer could generate robust signals across all targets. All polymerase signals were normalized to appropriate positive and negative controls. All measurements were performed in triplicate, and the data are displayed as mean±s.d.

FIG. 2 shows the inhibitory efficiency of the nanostructure with and without stabilizer domain. The signal of the nanostructure with (top and left curve) or without (bottom and right curve) stabilizer domain, for various variable region lengths. With a 6-bp stabilizer domain, the duplexed portion of the target-specific strand of the nanostructure could be short and still bind to and inhibit DNA polymerase enzyme, as seen by the decreasing signal. All measurements were performed in triplicate, and the data are displayed as mean±s.d.

FIG. 3 shows the thermodynamic properties of the stabilizer domain on the nanostructure. Different stabilizer domains with varying melting temperature (Tm)—high (dark grey), medium (grey) and low (light grey)—were incorporated into the nanostructure. With a high-Tm stabilizer domain, the nanostructure is able to efficiently inhibit DNA polymerase enzyme activity, even at an elevated incubation temperature. All measurements were performed in triplicate, and the data are displayed as mean±s.d.

FIG. 4 shows the versatility of the DNA nanostructure of the invention. By adjusting the sequence in the variable region, as well as that in the stabilizer domain, we developed nine nanostructures for different types of RNA classes. Each nanostructure demonstrated a high target response efficiency (>80% signal once target is added) and a high stability in absence of target (<10% signal).

FIG. 5 shows the specificity of the DNA nanostructure. Experiments were carried out by incubating the nanostructure with a short target (left, 18 bases) or a long target (right, 40 bases). In comparison to its signal with fully complementary targets, the nanostructure demonstrated a much lower signal, when being treated with respective single base-mutation targets. All signals were normalized against that of perfectly complementary target as well as no-target control.

FIG. 6 shows the detection sensitivity of the nanostructure. Target nucleic acids were titrated and incubated with the nanostructure. The resultant enzymatic activity was measured when coupled with sensitive secondary signaling reaction to produce signal. The dotted line shows the limit of detection (3 s.d. greater than background signal). All measurements were performed in triplicate, and the data are displayed as mean±s.d

FIG. 7 shows a two-input logic circuit design. By varying the combination of DNA nanostructures and the relative ratio of different components (i.e., aptamer, target-specific strand, and polymerase) in each nanostructure (top schematic), we programmed the following logic computations:OR gate, AND gate, NOT gate, NOR gate, and NAND gate. All target combinations and their expected computational outputs are summarized in corresponding truth tables (middle). The observed signals (bottom) showed a good agreement with the expected outputs. All signals were normalized to appropriate positive and negative controls. All measurements were performed in triplicate, and the data are displayed as mean±s.d.

FIG. 8 shows a two-input equalizer design. By adjusting the amount of target specific strand, two-input equalizer logic gates were constructed. Shown are examples for (a) OR and (b) AND. For each gate designed, the components used to establish the configuration are illustrated (top). All target combinations and their expected computational outputs are summarized in corresponding truth tables (middle). The observed signals (bottom) showed a good agreement with the expected outputs. All signals were normalized to appropriate positive and negative controls. All measurements were performed in triplicate, and the data are displayed as mean±s.d.

FIG. 9 shows multi-input logic gates. By varying the combination of DNA nanostructures and the ratio of different components (i.e., aptamer, target specific strand, and polymerase) in each nanostructure (left schematic), we also programmed the following logic computations:(a) 6-input OR gate and (b) 6-input AND gate. All signals were normalized to appropriate positive and negative controls. Normalized signals above the detection threshold (3 s.d. higher than background signal) (horizontal dotted line) were considered as true signals. All measurements were performed in triplicate, and the data are displayed as mean±s.d.

FIG. 10 shows thresholding modules. By varying the combination of DNA nanostructures and the ratio of different components (i.e., aptamer, target specific strand, and polymerase) in each nanostructure (left schematic), we also programmed the following logic computations:(a) 3/6 and (b) 5/6 thresholding module. All signals were normalized to appropriate positive and negative controls. Normalized signals above the detection threshold (3 s.d. higher than background signal)(horizontal dotted line) were considered as true signals. All measurements were performed in triplicate, and the data are displayed as mean±s.d.

FIG. 11 shows a clinical application for NSCLC diagnostics. (a) Quantification of individual RNA markers in clinical plasma samples. Lung cancer and normal samples were obtained from individual patients (n=20). (b) Schema of the operational processes using the computer classifier and molecular classifier for clinical NSCLC diagnostics. (Left) Computer classifier data processing using singleplex biomarker detection, to distinguish the risk of having NSCLC from the circulating RNAs. (Right) Molecular classifier of the same RNA samples in a single reaction that analyzes all 5 RNA markers simultaneously in a similar operational manner as the computer classifier. Normal controls are on the left and cancer patients are on the right in the risk score data graph. Difference between the normal and cancer population risk scores were calculated using student's t test. (**** indicates p<0.0005).

### DETAILED DESCRIPTION OF THE INVENTION

With rational design, the nanostructures are developed to harbor different functional stability to recognize a wide variety of RNA targets (of different lengths and sequence content), including single base mutations. Moreover, the system is compatible with different readout modalities, in different environments, for enhanced portability. Further, the system is capable of multiplexed molecular logic operations in a one-pot reaction. The system is able to:
i) equalize different target concentration,
ii) perform multiplier functions (multiple targets),
iii) implement thresholding functionality, and
iv) operate at ambient temperature

Bibliographic references mentioned in the present specification are for convenience listed in the form of a list of references and added at the end of the Examples. The whole content of such bibliographic references is herein incorporated by reference.

### Definitions

Unless defined otherwise, all technical and scientific terms used herein have the same meaning as is commonly understood by one of skill in the art to which the invention belongs. Certain terms employed in the specification, examples and appended claims are collected here for convenience.

It must be noted that as used herein and in the appended claims, the singular forms “a,” “an,” and “the” include plural reference unless the context clearly dictates otherwise. Thus, for example, a reference to “a target sequence” includes a plurality of such target sequences, and a reference to “an enzyme” is a reference to one or more enzymes and equivalents thereof known to those skilled in the art, and so forth.

As used herein, the term “aptamer”, refers to single stranded DNA or RNA molecules. An aptamer is capable of binding various molecules with high affinity and specificity. For example, as used herein, in the absence of target DNA, the DNA aptamer binds strongly with the polymerase to inhibit polymerase activity.

The phrases “nucleic acid” or “nucleic acid sequence,” as used herein, refer to an oligonucleotide, nucleotide, polynucleotide, or any fragment thereof, to DNA or RNA of genomic or synthetic origin which may be single-stranded or double-stranded and may represent the sense or the antisense strand, to peptide nucleic acid (PNA), or to any DNA-like or RNA-like material.

As used herein, the term “oligonucleotide”, refers to a nucleic acid sequence of at least about 6 nucleotides to 60 nucleotides, preferably about 15 to 30 nucleotides, and most preferably about 20 to 25 nucleotides, which can be used in PCR amplification or in a hybridization assay or microarray. As used herein, the term “oligonucleotide” is substantially equivalent to the terms “amplimers,” “primers,” “oligomers,” and “probes,” as these terms are commonly defined in the art.

As used herein, the term “conserved sequence region” refers to a region of the DNA polymerase enzyme-specific aptamer that binds to inactivate DNA polymerase.

As used herein, the term “variable sequence region” refers to a region that comprises a segment that is complementary to a portion of a target-specific inverter oligonucleotide.

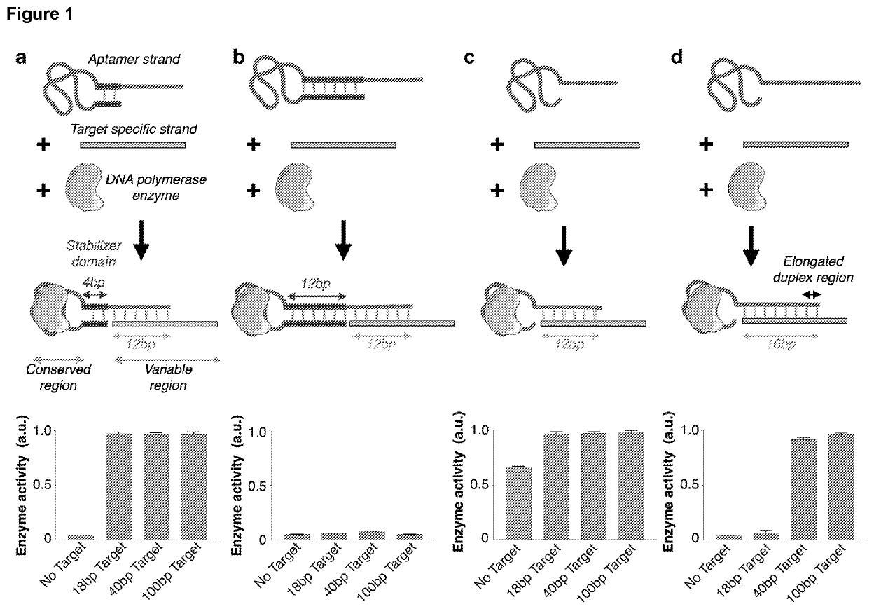

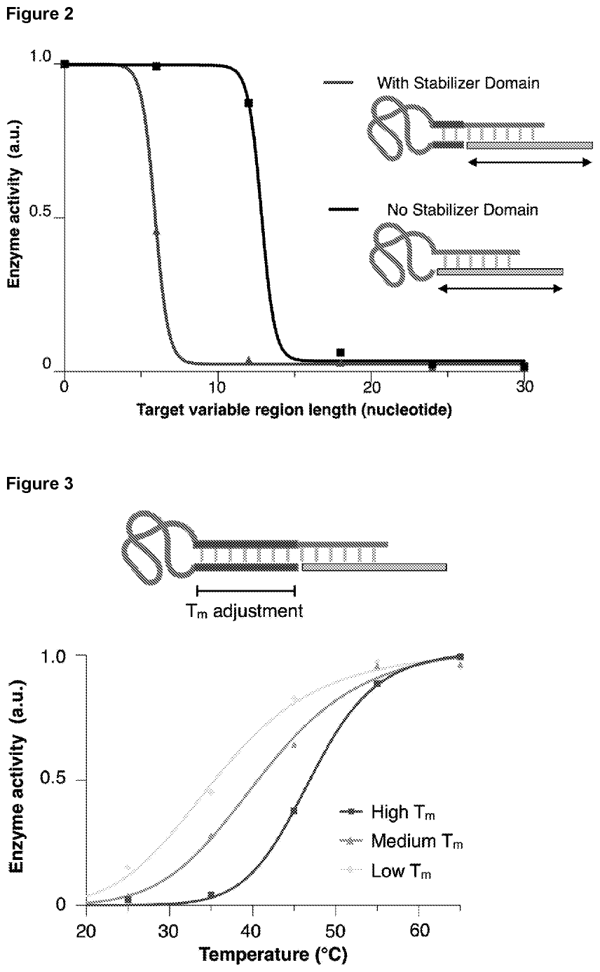

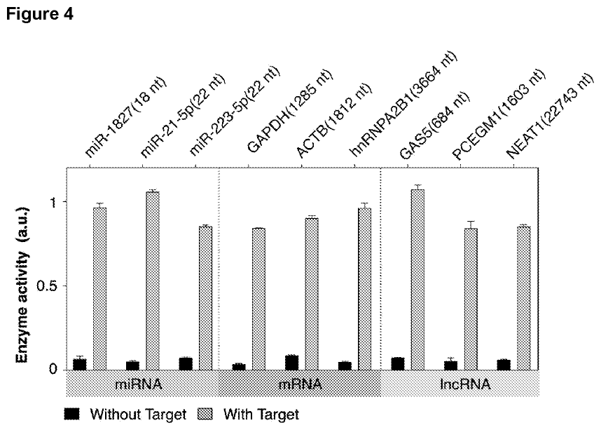

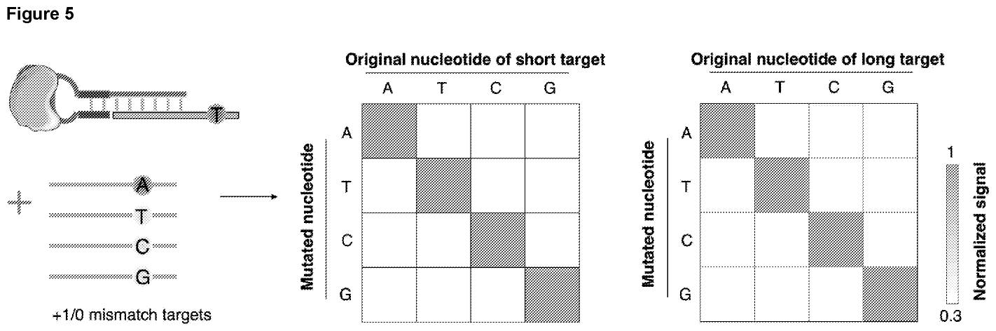

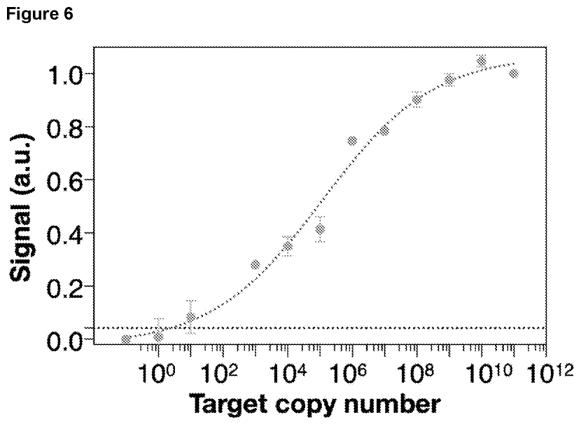

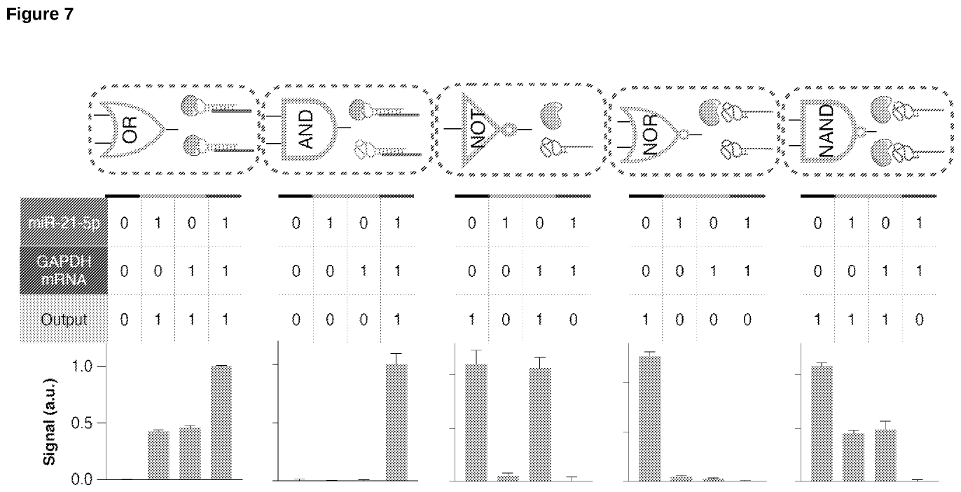

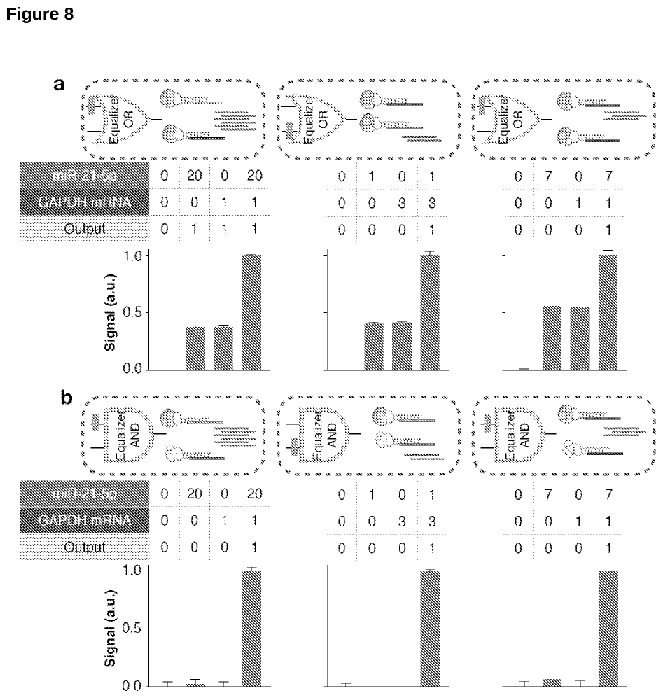

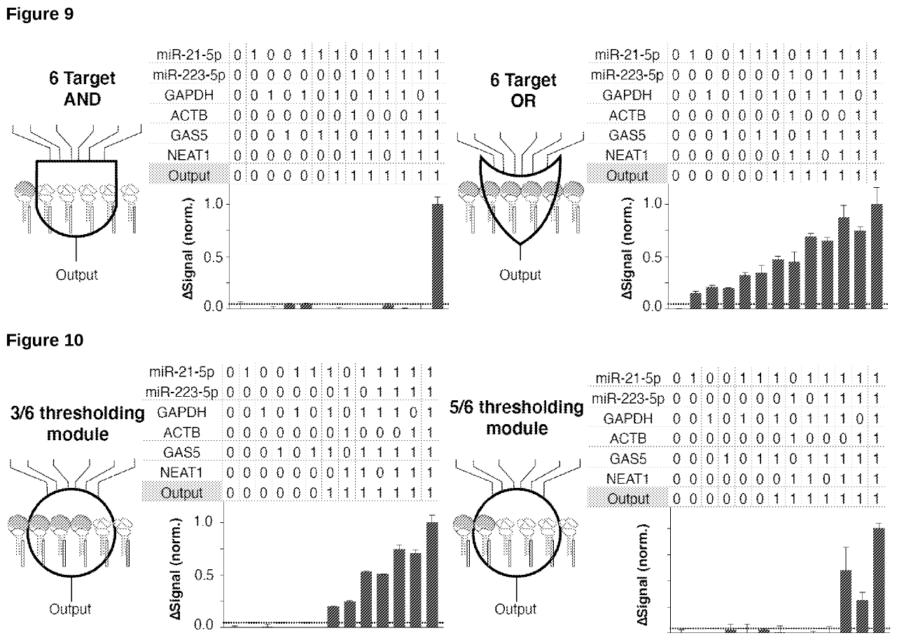

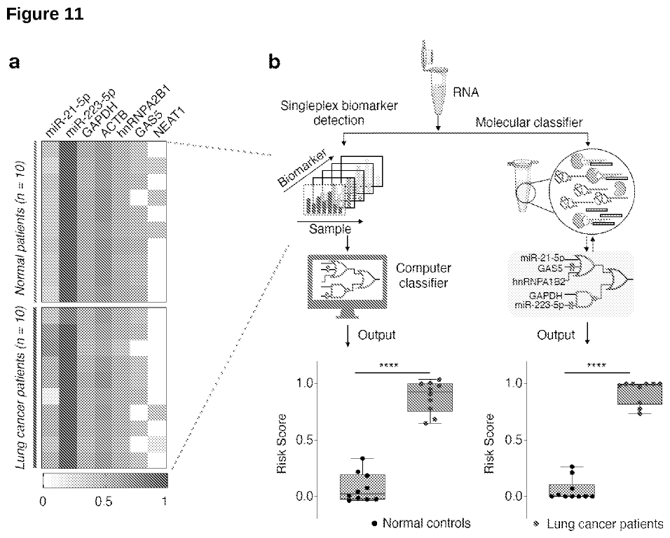

As used herein, the term “duplex stabilizer region” or “duplex stabilizer domain” refers to a region of the DNA polymerase enzyme-specific aptamer that lies between the conserved aptamer sequence region and the variable sequence region, wherein variation of the length and/or composition of the duplex stabilizer region can vary the conformational stability of the nanostructure. A pictorial representation of the conserved, stabilizer and variable regions is shown in FIG. 1.

As used herein, the term “inverter sequence” or “inverter oligonucleotide” refers to an oligonucleotide which is complementary to a target nucleic acid sequence, and which a portion is involved in forming a duplex with the variable sequence region of the aptamer and a portion forms an overhang and is involved in duplexing with the target nucleic acid.

Herein it is shown that a short stabilizer region renders the aptamer more sensitive to target sequences than a longer stabilizer domain (see FIGS. 1a and 1b). Herein it is shown that, in the absence of a stabilizer domain, as the duplex region is elongated the nanostructure is more stable to short targets (see FIGS. 1c and 1d).

The term “sample,” is used herein in its broadest sense. For example, a biological sample may be suspected of containing RNA sequences corresponding to a disease. The sample may comprise a bodily fluid; an extract from a cell, chromosome, organelle, or membrane isolated from a cell; a cell; genomic DNA, RNA, or cDNA (in solution or bound to a solid support); a tissue; a tissue print; and the like. An example shown herein is an isolated blood sample suspected of containing RNA sequences corresponding to NSCLC markers, such as SEQ ID Nos:28, 30, 38 and 40.

It would be understood that oligonucleotides used in the present invention may be structurally and/or chemically modified to, for example, prolong their activity in samples potentially containing nucleases, during performance of methods of the invention, or to improve shelf-life in a kit. Thus, the aptamer and/or inverter or any oligonucleotide primers or probes used according to the invention may be chemically modified. In some embodiments, said structural and/or chemical modifications include the addition of tags, such as fluorescent tags, radioactive tags, biotin, a 5′ tail, the addition of phosphorothioate (PS) bonds, 2′-O-Methyl modifications and/or phosphoramidite C3 Spacers during synthesis.

As used herein, the term “comprising” or “including” is to be interpreted as specifying the presence of the stated features, integers, steps or components as referred to, but does not preclude the presence or addition of one or more features, integers, steps or components, or groups thereof. However, in context with the present disclosure, the term “comprising” or “including” also includes “consisting of”. The variations of the word “comprising”, such as “comprise” and “comprises”, and “including”, such as “include” and “includes”, have correspondingly varied meanings.

### EXAMPLES

Standard molecular biology techniques known in the art and not specifically described were generally followed as described in Green and Sambrook, Molecular Cloning:A Laboratory Manual, Cold Spring Harbour Laboratory, New York (2012).

### Example 1

### Methods

### Nanostructure Assembly

For preparation of the recognition nanostructure, we incubated an optimized ratio of the DNA oligonucleotide components (IDT) at 95° C. for 5 min in a buffer of 50 mM NaCl, 1.5 mM MgCl₂, and 50 mM Tris—HCl buffer (pH 8.5). The mixture was then slowly cooled to room temperature at 0.1° C./s upon which a suitable molar amount of Taq DNA polymerase (Promega) was added to form the lock nanostructure.

### Nanostructure Characterization

To characterize the response of different recognition DNA nanostructures in the presence of nucleic acid targets, synthetic oligonucleotides matching target sequences (IDT) were used. Titrations of target were added to the DNA lock nanostructure solution and polymerase activity was determined by (A) using a 5′ Exonuclease degradation of pre-annealed Taqman probe measured over time to measure the kinetics of polymerase release or (B) using elongation of pre-annealed signaling nanostructure immobilized on an electrode and measuring peroxidase activity before and after sample incubation.

All sequences can be found in Tables

### RNA Extraction

Total RNA was isolated from samples using RNeasy kit (Qiagen), according to the manufacturer's protocol. Samples were diluted in RLT lysis buffer, and vortexed for 1 minute at maximum speed. 1 volume of 70% ethanol is added to the sample and mixed well by vortexing. This mixture is added onto a spin column and centrifuged at maximum speed for 15 seconds. On column DNase digestion was performed before column was rinsed using RPE buffer. RNA was eluted in nuclease-free water. The quality and quantity of extracted RNA were measured with a spectrophotometer (Thermo Scientific) and stored at −80° C. before being used.

### Clinical Measurements

1 μl of the extracted RNA was mixed into the molecular classifier and then incubated at room temperature on the electrode and measured as described previously. The data was analyzed using Principal Component Analysis to reduce the marker dimensions and plot the separation of cases and controls. The markers which made up the largest weights for the principal components with the best separation were used in a multiple logistic regression model to appropriately weigh the marker signals for building a classifier. The classification performance was determined using leave-one-out analysis to make predictions for all patients.

Using the logistic model, we designed a computational circuit using the same five markers that would encode the same algorithm. Equalizer gates were adjusted to the different basal RNA levels, and to produce outputs in proportion to the marker weights in the logistic model. Based on the direction of the weights, either positive (regular gate) or negative (NOT gate) logic gates were added into the circuit. The normalized output signals from the molecular classifier were used as the risk scores.

### Example 2

### Nanostructure Design

Each nanostructure comprises a polymerase-specific DNA aptamer, a target-specific DNA strand and a DNA polymerase enzyme. In comparison to our previously developed nanostructure [Ho, N. R. Y. et al., Nat Commun 9:3238 (2018)], we engineered a stabilizer domain in the variable region, that lies between the conserved aptamer sequence and the target-specific strand (FIG. 1). This stabilizer domain helps to maintain the nanostructure's conformational stability, even when the target-specific strand is short or thermodynamically unstable, thereby enabling versatile activation by short and long nucleic acid targets alike (FIG. 1a). Notably, a long and strong stabilizer domain creates such a stable nanostructure that it cannot be activated even in the presence of the target (FIG. 1b). Without the stabilizer domain, the nanostructure is either unstable and cannot fully silence the enzyme activity (FIG. 1c, short duplex) or only responsive to long targets but not to short targets (FIG. 1d, long duplex). Sequences used are found in Table 1.

TABLE 1
FIG. 1 sequences

| | Stabilizer domain | SEQ |
|---|---|---|
| | sequence | ID NO: |
| Aptamer (no | GTTGCGCAGCCTCAATGT | 1 |
| stabilization | ACAGTATTG | |
| domain) | | |
| Aptamer (no | AACAGTTGCGCAGCCTCAAT | 2 |
| stabilization | GTACAGTATTG | |
| domain) | | |
| elongated duplex | | |
| Aptamer (4 bp | GTTGCGCAGCCTACGCCAAT | 3 |
| stabilization | GTACAGTATTGGCGT | |
| domain) | | |
| Aptamer (12 bp | GTTGCGCAGCCTCGCAGAGG | 4 |
| stabilization | TGAGCAATGTACAGTATTGC | |
| domain) | TCACCTCTGCG | |
| 18 bp Target | AGGCTGCGCAACTGTTGG | 5 |
| Specific | | |
| Strand | | |
| 40 bp Target | AGGCTGCGCAACTGTTGGGAA | 6 |
| Specific | GGGCGATCGGTGCGGGCCT | |
| Strand | | |
| 100 bp Target | AGGCTGCGCAACTGTTGGGAA | 7 |
| Specific | GGGCGATCGGTGCGGGCCTCT | |
| Strand | TCGCTATTACGCCAGCTGGCG | |
| | AAAGGGGGATGTGCTGCAAGG | |
| | CGATTAAGTTGGGTAA | |
| 18 bp Target | CCAACAGTTGCGCAGCCT | 8 |
| 40 bp Target | AGGCCCGCACCGATCGCCCTT | 9 |
| | CCCAACAGTTGCGCAGCCT | |
| 100 bp Target | TTACCCAACTTAATCGCCTT | 10 |
| | GCAGCACATCCCCCTTTCGC | |
| | CAGCTGGCGTAATAGCGAAG | |
| | AGGCCCGCACCGATCGCCCT | |
| | TCCCAACAGTTGCGCAGCCT | |

### Example 3

### Tunable Stability

The incorporation of the stabilizer domain thus greatly expands the stability and versatility of the nanostructure. The thermodynamic properties of the stabilizer domain can be accurately predicted based on various models of Gibbs free energy of DNA hybridization (ΔG°) [Tulpan, D., Andronescu, M. & Leger, S. BMC Bioinformatics 11:105 (2010)]. By adding a 6-bp stabilizer domain with the sequence ACTGGC, the nanostructure is able to form a stable enzyme-aptamer complex with only a 12-bp duplex with the target specific strand. The nanostructure without the stabilizer domain, however, needed an 18-bp duplex with the target specific strand to form a stable enzyme-aptamer complex (FIG. 2 and Table 2).

TABLE 2
FIG. 2 sequences

| | | SEQ ID |
|---|---|---|
| | Stabilizer domain sequence | NO: |
| Aptamer (no | CAATGTACAGTATTG | 11 |
| stabilization domain) | | |
| (0 bp duplex with target | | |
| specific strand) | | |
| Aptamer (no | CAGCCTCAATGTACAGTATTG | 12 |
| stabilization domain) | | |
| (6 bp duplex with target | | |
| specific strand) | | |
| Aptamer (no | GTTGCGCAGCCTCAATGTACAGTATTG | 13 |
| stabilization domain) | | |
| (12 bp duplex with | | |
| target specific strand) | | |
| Aptamer (no | CCAACAGTTGCGCAGCCTCAATGTACAGTATTG | 14 |
| stabilization domain) | | |
| (18 bp duplex with | | |
| target specific strand) | | |
| Aptamer (no | CCCTTCCCAACAGTTGCGCAGCCT | 15 |
| stabilization domain) | CAATGTACAGTATTG | |
| (24 bp duplex with | | |
| target specific strand) | | |
| Aptamer (no | CGATCGCCCTTCCCAACAGTTGCGC | 16 |
| stabilization domain) | AGCCTCAATGTACAGTATTG | |
| (30 bp duplex with | | |
| target specific strand) | | |
| Aptamer (6 bp | ACTGGCCAATGTACAGTATTGGCCA | 17 |
| stabilization domain) | GT | |
| (0 bp duplex with target | | |
| specific strand) | | |
| Aptamer (6 bp | CAGCCTACTGGCCAATGTACAGTATTGGCCAGT | 18 |
| stabilization domain) | | |
| (6 bp duplex with target | | |
| specific strand) | | |
| Aptamer (6 bp | GTTGCGCAGCCTACTGGCCAATGTACAGTATT | 19 |
| stabilization domain) | GGCCAGT | |
| (12 bp duplex with | | |
| target specific strand) | | |
| Aptamer (6 bp | CCAACAGTTGCGCAGCCTACTGGCCAATGTACAG | 20 |
| stabilization domain) | TATTGGCCAGT | |
| (18 bp duplex with | | |
| target specific strand) | | |
| Aptamer (6 bp | CCCTTCCCAACAGTTGCGCAGCCTACTGGCCAAT | 21 |
| stabilization domain) | GTACAGTATTGGCCAGT | |
| (24 bp duplex with | | |
| target specific strand) | | |
| Aptamer (6 bp | CGATCGCCCTTCCCAACAGTTGCGCAGCCTACTG | 22 |
| stabilization domain) | GCCAATGTACAGTATTGGCCAGT | |
| (30 bp duplex with | | |
| target specific strand) | | |
| Target Specific Strand | AGGCTGCGCAACTGTTGGGAAGGGCGATCGGTGC | 23 |
| | GGGCCT | |

The stabilizer domain can also be tuned to design DNA-enzyme nanostructures that respond with different thermodynamic profiles. Using sequences which have more inherent stability (more negative b.G0), we can design nanostructures that are more resistant to factors that can negatively affect its stability such as (1) duplex length between aptamer and target specific strand (shorter=less stable), (2) sequence composition of the duplex between aptamer and target specific strand (low GC content=less stable), (3) temperature (higher=less stable), (4) salt concentration (less positive monovalent or divalent salts=less stable), (5) pH (outside of range 6-B=less stable), and (6) chemicals that impact hybridization such as formamide or spermine (depending on chemical; formamide:higher=less stable, spermine:lower=less stable). Using a short 4-bp stabilization domain with high AT content (low Tm), we created a stable nanostructure at 20° C., but which was quickly destabilized at higher temperatures. By changing the content of the stabilizer domain to one with a higher GC content while maintaining the length the same (medium Tm), the nanostructure showed greater stability to increased temperatures. Lastly, by increasing both the length and increasing the GC content of the stabilizer domain (high Tm), we created a nanostructure that was most resistant to increased temperatures, fully stable even at 35° C. (FIG. 3 and Table 3).

TABLE 3
FIG. 3 sequences

| | Stabilizer | SEQ |
|---|---|---|
| | domain | ID |
| | sequence | NO: |
| Low Tm | GTTGCGCAGCCTTTATC | 24 |
| Aptamer | AATGTACAGTATGATAA | |
| Medium Tm | GTTGCGCAGCCTCGACC | 25 |
| Aptamer | AATGTACAGTATTGGTC | |
| | G | |
| High Tm | GTTGCGCAGCCTGACAC | 26 |
| Aptamer | GACCAATGTACAGTATT | |
| | GGTCGTGTC | |
| Target | AGGCTGCGCAACTGTTG | 27 |
| Specific | G | |
| Strand | | |

### Example 4

### Performance Evaluation

The stabilizer domain can be tuned to normalize the performance of the nanostructures against different target sequences that have a wide variety of ΔG°. We designed a variety of nanostructures against different types of RNA targets, by tuning the stabilizer domain in each one to ensure the nanostructures would be within ±2 kcal/mol ΔG° of each other (Table 4). As a result, we were able to develop unique nanostructures for a wide variety of RNA targets (e.g., miRNA, mRNA, IncRNA) with a similar performance profile (FIG. 4).

The incorporation of the stabilizer domain further enables the detection of single-base mismatches with high specificity. Without the stabilizer domain, as in our previous design, we are limited to selecting target sequences and single nucleotide positions which create a large thermodynamic gap when mutated. However, through careful design of the stabilizer domain, we can maximize the thermodynamic gap for different types of single-base mismatches; this not only enhances the nanostructure's ability to decipher mismatches, at a high resolution (i.e., single-base mutations), but also enables it to accommodate a wide variety of target sequences (FIG. 5 and Table 5).

TABLE 4

| | Stabilizer domain | SEQ | | | SEQ |
|---|---|---|---|---|---|
| | sequence | ID NO: | | | ID NO: |
| miR-21-5p | GACTGATGTTGACGCCAATGTA | 28 | miR-21 | TCAACATCAGTCTGATAA | 29 |
| Aptamer | CAGTATTGGCG | | Specific Strand | | |
| miR-223-5p | ACAAGCTGAGTTCGCCAATGT | 30 | miR-223 | AACTCAGCTTGTCAAATA | 31 |
| Aptamer | ACAGTATTGGCG | | Specific Strand | | |
| miR-1827 | AGTAGATTGAATCGGACAATG | 32 | miR-1827 | ATTCAATCTACTGCCTCA | 33 |
| Aptamer | TACAGTATTGTCCG | | Specific Strand | | |
| GAPDH | TTCTCAAGACGGAATCAATGTA | 34 | GAPDH | CCGTCTTGAGAAACCTGC | 35 |
| Aptamer | CAGTATTGATT | | Specific Strand | | |
| ACTB | ACGCAACTAAGTGACCAATGTA | 36 | ACTB Specific | ACTTAGTTGCGTTACACC | 37 |
| Aptamer | CAGTATTGGTC | | Strand | | |
| hnRNPA2B1 | TGCCTATCAGTATGACCAATGT | 38 | hnRNPA2B1 | TACTGATAGGCAGTCTGG | 39 |
| Aptamer | ACAGTATTGGTCA | | Specific Strand | | |
| GAS5 | TGTCTTCATGTCTAGCCAATGT | 40 | GAS5 Specific | GACATGAAGACAGTTCCT | 41 |
| Aptamer | ACAGTATTGGCTA | | Strand | | |
| PCGEM1 | CCTCAGAAATCTCGGCAATGTA | 42 | PCEGM1 | AGATTTCTGAGGGGAATT | 43 |
| Aptamer | CAGTATTGCCG | | Specific Strand | | |
| NEAT1 | TTAGCGCCAAACTAGCAATGTA | 44 | NEAT1 | GTTTGGCGCTAAACTCTT | 45 |
| Aptamer | CAGTATTGCTA | | Specific Strand | | |

| | | |
|---|---|---|
| miR-21-5p NOT | TCAGTCTGATAACGCCAATGTA | 46 |
| Aptamer | CAGTATTGGCG | |
| miR-223-5p NOT | GCTTGTCAAATACGCCAATGTA | 47 |
| Aptamer | CAGTATTGGCG | |
| miR-1827 NOT | TCTACTGCCTCACGGACAATGT | 48 |
| Aptamer | ACAGTATTGTCCG | |
| GAPDH NOT | TGAGAAACCTGCAATCAATGTA | 49 |
| Aptamer | CAGTATTGATT | |
| ACTB NOT | TTGCGTTACACCGACCAATGTA | 50 |
| Aptamer | CAGTATTGGTC | |
| hnRNPA2B1 NOT | TAGGCAGTCTGGTGACCAATGT | 51 |
| Aptamer | ACAGTATTGGTCA | |
| GAS5 NOT | AAGACAGTTCCTTAGCCAATGT | 52 |
| Aptamer | ACAGTATTGGCTA | |
| PCGEM1 NOT | CTGAGGGGAATTCGGCAATGTA | 53 |
| Aptamer | CAGTATTGCCG | |
| NEAT1 NOT | CGCTAAACTCTTTAGCAATGTA | 54 |
| Aptamer | CAGTATTGCTA | |

TABLE 5
Mismatch characterization sequences

| | | SEQ ID |
|---|---|---|
| | Stabilizer domain sequence | NO: |
| Aptamer (short target) | AGTAGATTGAATCGCCAATGTACAGTATTGGCG | 55 |
| Short Target Specific | ATTCAATCTACTGTCTCA | 56 |
| Strand (originally A) | | |
| Short Target Specific | ATTCAATCTACTGGCTCA | 57 |
| Strand (originally C) | | |
| Short Target Specific | ATTCAATCTACTGACTCA | 58 |
| Strand (originally T) | | |
| Short Target Specific | ATTCAATCTACTGCCTCA | 59 |
| Strand (originally G) | | |
| Short Target (A) | TGAGACAGTAGATTGAAT | 60 |
| Short Target (C) | TGAGCCAGTAGATTGAAT | 61 |
| Short Target (T) | TGAGTCAGTAGATTGAAT | 62 |
| Short Target (G) | TGAGGCAGTAGATTGAAT | 63 |
| Aptamer (long target) | TGCCTATCAGTACGCCAATGTACAGTATTGGCG | 64 |
| Long Target Specific | TACTGATAGGCAGTCTGT | 65 |
| Strand (originally A) | | |
| Long Target Specific | TACTGATAGGCAGTCTGG | 66 |
| Strand (originally C) | | |
| Long Target Specific | TACTGATAGGCAGTCTGA | 67 |
| Strand (originally T) | | |
| Long Target Specific | TACTGATAGGCAGTCTGC | 68 |
| Strand (originally G) | | |
| Long Target (A) | CTTTTCTTTACAGACTGCCTATCAGTAATTATCTCAATGG | 69 |
| Long Target (C) | CTTTTCTTTCCAGACTGCCTATCAGTAATTATCTCAATGG | 70 |
| Long Target (T) | CTTTTCTTTTCAGACTGCCTATCAGTAATTATCTCAATGG | 71 |
| Long Target (G) | CTTTTCTTTGCAGACTGCCTATCAGTAATTATCTCAATGG | 72 |

When coupled with downstream assays that amplify and transduce the enzyme activity signal (e.g., additional enzymatic recruitment [PCT/SG2021/050194; WO 2020/009660], the new nanostructure enables sensitive detection of target nucleic acids (FIG. 6).

### Example 5

### Multiplexed Molecular Logic Operations

The nanostructures can be combined into a single pot reaction and perform multiplexed molecular logic operations. This is controlled by titrating the amount of each constituent component used in the reaction (i.e., polymerase, aptamer strand and target-specific strand). For example, by mixing the polymerase enzyme, aptamer strand 1, target-specific strand 1, aptamer strand 2, and target-specific strand 2 in different ratios, the one-pot assay produces OR (2:1:1:1:1), AND (1:1:1:1:1), NOT (1:1:0:0:0 or 1:0:0:1:0), NOR (1:1:0:1:0), or NAND (2:1:0:1:0) logic functions (FIG. 7).

By further varying the amount of the target-specific strand, we can further tune the sensitivity of the nanostructures to respond differently to the same amount of target or to respond identically to different amounts of target. In essence, this acts as a molecular sink that binds with excess target copies to equalize the signals arising from different amounts of targets. When mixed in the logic function titration ratios, an equalizer logic function can be created (FIG. 8). This can be useful in the context of measuring gene expression levels of different targets, where each gene target has a different basal level of expression.

Furthermore, the logic functions can be extended beyond two targets. By mixing multiple nanostructures in the same reaction, we can create a multi-target assay that responds differently to the same target input (FIG. 9). Moreover, by further tuning the combination of the nanostructures in a one-pot reaction, thresholds can be set to measure the number of targets whose expression exceeds the set threshold (FIG. 10).

### Example 6

### Clinical Application

We quantified the amount of circulating RNA markers from patient sera (10 NSCLC and 10 control samples) (FIG. 11a). Based on individual biomarker expression, we built a machine learning model that used 5 different-type RNA markers (i.e., miR-21-5p, miR-223-5p, GAPDH, hnRNPA1B2, and GAS5). The computed RNA signature was able to accurately distinguish between the lung cancer patients and normal controls based on the RNA signature (FIG. 11b, left).

We next built a 5-marker multiplexed molecular logic operation that mimicked the machine learning model in a chemical reaction. Measurements using this molecular classifier (FIG. 11b, right) directly on patient circulating RNA had a comparable performance with the computational classifier which used individual marker measurements. This showcases the use of molecular logic gates to create chemical algorithms which can be used to quantify a panel of different markers in a one-pot reaction to accurately diagnose patients.

Any listing or discussion of an apparently prior-published document in this specification should not necessarily be taken as an acknowledgement that such document is part of the state of the art or is common general knowledge.

### References

1. Ho, N. R. Y. et al. Visual and modular detection of pathogen nucleic acids with enzyme-DNA molecular complexes. Nat Commun 9:3238 (2018).
2. Tulpan, D., Andronescu, M. & Leger, S. Free energy estimation of short DNA duplex hybridizations. BMC Bioinformatics 11:105 (2010).
3. PCT/SG2021/050194 Shao, H., Ho, N. R. Y., Sundah, N. R., Liu, Y. & Chen, Y. Responsive, Catalytic Nucleic Nanostructures. National University of Singapore, Agency for Science, Technology and Research, Singapore.
4. WO 2020/009660 Shao, H. & Ho, N. R. Y. Visual and Modular Detection of Nucleic Acids with Enzyme-Assisted Nanotechnology. National University of Singapore, Agency for Science, Technology and Research Singapore, (2020).

1. A recognition nanostructure comprising a DNA polymerase enzyme-specific DNA aptamer having a conserved sequence region that binds to inactivate DNA polymerase, a variable sequence region comprising a segment that is complementary to a portion of a target-specific inverter oligonucleotide, and a duplex stabilizer region that lies between the conserved aptamer sequence region and the variable sequence region,
wherein variation of the length and/or composition of the duplex stabilizer region can vary the conformational stability of the nanostructure.

2. The recognition nanostructure of claim 1, wherein the variable sequence region is at least 8 nucleotides in length.

3. The recognition nanostructure of claim 1, further comprising a target-specific inverter oligonucleotide, wherein a portion of the target-specific inverter oligonucleotide forms a duplex with the variable sequence region and a portion of the target-specific inverter oligonucleotide forms an overhang of at least 4 nucleotides.

4. The recognition nanostructure of claim 1, wherein increasing the length and/or the GC content of the duplex stabilizer domain increases the conformational stability of the nanostructure, thereby altering;
i) target compatibility; and/or
ii) kinetics of enzyme activation; and/or
iii) ability to measure inputs.

5. The recognition nanostructure of claim 1, wherein the duplex stabilizer domain length is in the range of 1 to 20 nucleic acids.

6. The recognition nanostructure of claim 1, wherein the duplex stabilizer region:
i) comprises a nucleic acid sequence set forth in any of Tables 1 to 4;
ii) comprises the nucleic acid sequence selected from the group consisting of SEQ ID NO: 3, SEQ ID NO: 4, SEQ ID NO: 17, SEQ ID NO: 18, SEQ ID NO: 19, SEQ ID NO: 20, SEQ ID NO: 21, SEQ ID NO: 22, SEQ ID NO: 24, SEQ ID NO: 25, SEQ ID NO:26, SEQ ID NO: 28, SEQ ID NO: 30, SEQ ID NO: 32, SEQ ID NO: 34, SEQ ID NO:36, SEQ ID NO: 38, SEQ ID NO: 40, SEQ ID NO: 42, SEQ ID NO: 44, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53 and SEQ ID NO: 54; or
iii) consists of the nucleic acid sequence selected from the group consisting of SEQ ID NO:28, SEQ ID NO: 30, SEQ ID NO: 32, SEQ ID NO: 34, SEQ ID NO: 36, SEQ ID NO:38, SEQ ID NO: 40, SEQ ID NO: 42, SEQ ID NO: 44, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53 and SEQ ID NO: 54.

7. (canceled)

8. (canceled)

9. A method of detecting target nucleic acids in a sample, comprising the steps of:
(a) providing a sample comprising nucleic acid;
(b) providing a composition comprising a DNA polymerase enzyme, a recognition nanostructure defined in claim 1, and a target-specific inverter oligonucleotide; or
(c) providing a composition comprising a DNA polymerase enzyme and the recognition nanostructure that further comprises a target-specific inverter oligonucleotide, wherein a portion of the target-specific inverter oligonucleotide forms a duplex with the variable sequence region and a portion of the target-specific inverter oligonucleotide forms an overhang of at least 4 nucleotides;
(d) contacting the sample comprising nucleic acid with the composition of (b) or (c), wherein target nucleic acid binding to the inverter oligonucleotide destabilizes the recognition nanostructure, thereby releasing the DNA polymerase enzyme from inhibition by the DNA aptamer;
(e) detecting DNA polymerase enzyme activity, wherein the intensity of activity indicates the presence of target nucleic acid in the sample.

10. A method of diagnosing a disease in a subject, comprising the steps of:
(a) providing a sample comprising nucleic acid;
(b) providing a composition comprising a DNA polymerase enzyme, a recognition nanostructure defined in claim 1, and a target-specific inverter oligonucleotide; or
(c) providing a composition comprising a DNA polymerase enzyme and the recognition nanostructure that further comprises a target-specific inverter oligonucleotide, wherein a portion of the target-specific inverter oligonucleotide forms a duplex with the variable sequence region and a portion of the target-specific inverter oligonucleotide forms an overhang of at least 4 nucleotides;
(d) contacting the sample comprising nucleic acid with the composition of (b) or (c), wherein target nucleic acid binding to the inverter oligonucleotide destabilizes the recognition nanostructure, thereby releasing the DNA polymerase enzyme from inhibition by the DNA aptamer;
(e) detecting DNA polymerase enzyme activity, wherein the intensity of activity indicates the presence of target nucleic acid in the sample;
(f) diagnosing the subject with the disease when presence of target nucleic acid in the sample is detected.

11. The method of claim 10, wherein the inverter oligonucleotide is about 18 to 45 nucleotides in length.

12. The method according to claim 10, further comprising providing one or more additional recognition nanostructures complementary to one or more target nucleic acids different from the target nucleic acid of a first recognition nanostructure in the sample, for multiplex detection.

13. The method of claim 12, wherein the each of the recognition nanostructures comprises a combination of DNA aptamer:inverter oligonucleotide:DNA polymerase enzyme in a ratio to form a logic gate selected from the group consisting of AND, OR, NOT, NAND and NOR.

14. The method of claim 13, wherein the combination of DNA aptamer:inverter oligonucleotide:DNA polymerase enzyme ratio of each recognition nanostructure is selected from the group consisting of:
(i) two nanostructures each having 1:1:0.5 DNA aptamer:inverter oligonucleotide:DNA polymerase enzyme ratio to form a AND logic gate;
(ii) two nanostructures each having 1:1:1 DNA aptamer:inverter oligonucleotide:DNA polymerase enzyme ratio to form a OR logic gate;
(iii) one nanostructure having 1:0:1 to form a NOT logic gate;
(iv) two nanostructures each having 1:0:1 DNA aptamer:inverter oligonucleotide:DNA polymerase enzyme ratio to form a NAND gate; and
(v) two nanostructures each having 1:0:0.5 DNA aptamer:inverter oligonucleotide:DNA polymerase enzyme ratio to form a NOR gate.

15. The method of claim 13, wherein the relative amount of inverter oligonucleotide in the combination of DNA aptamer:inverter oligonucleotide:DNA polymerase enzyme ratio of each recognition nanostructure is adjusted to equalize the DNA polymerase enzyme activity when there are differences in levels of a plurality of targets in the sample.

16. The method of claim 13, wherein the combination of DNA nanostructures and the ratio of the DNA aptamer, inverter oligonucleotide, and DNA polymerase in each nanostructure is varied to provide (a) a multi-input OR gate or (b) a multi-input AND gate.

17. The method of claim 13, wherein the combination of DNA nanostructures and the ratio of the DNA aptamer, inverter oligonucleotide, and DNA polymerase in each nanostructure is varied to provide a threshold level of detection of a plurality of targets in a sample.

18. The method of claim 12, wherein the disease is NSCLC and the recognition nanostructures detect a plurality of RNA species selected from the group consisting of miR-21-5p, miR-223-5p, GAPDH, hnRNPA2B1 and GAS5 or combinations thereof.

19. The method according to claim 18, wherein the recognition nanostructure oligonucleotide sequences are selected from the group consisting of SEQ ID NO: 28, SEQ ID NO: 30, SEQ ID NO: 34, SEQ ID NO: 38 and SEQ ID NO: 40.

20. A device comprising:
(i) at least one DNA polymerase enzyme and at least one recognition nanostructure, as defined in claim 1, at a 1^{st} location;
(ii) signaling nanostructures comprising a self-priming portion responsive to active DNA polymerase enzyme, attached at a 2^{nd} location; and
(iii) an intermediate stage for mixing of said recognition nanostructures with sample nucleic acid to release active enzyme to said 2^{nd} location.

21. The device of claim 20, selected from a microfluidic device and a lateral flow device.

22. The device of claim 21, wherein the device is a microfluidic device comprising:
(i) a common signaling cartridge configured to receive one or more assay cassettes, wherein the cartridge comprises a base with membranes embedded to immobilize signaling nanostructures, and a common outlet which makes fluid connection with said 2^{nd} location in each of the one or more assay cassettes;
(ii) one or more assay cassettes each comprising, at a 1^{st} location, an inlet and at least one DNA polymerase enzyme and at least one recognition nanostructure; an intermediate stage microchannel in fluid connection between the 1^{st} and 2^{nd} locations, for mixing of said detection nanostructures with sample nucleic acid to release active enzyme to said 2^{nd} location;
wherein, when the device is assembled and in use, there is fluidic flow from the sample inlet to the common outlet, actuated by a withdrawal septum.

23. A nucleic acid detection kit comprising;
(a) a composition comprising at least one DNA polymerase enzyme and at least one recognition nanostructure defined in claim 3;
(b) a signaling nanostructure that is reactive to active DNA polymerase enzyme, wherein the signaling nanostructure comprises a self-priming portion responsive to the DNA polymerase enzyme; or
(c) labelled nucleotides (dNTPs) and signal development reagents, wherein active DNA polymerase enzyme adds labelled nucleotides to the signaling nanostructure and the signal development reagents bind to the labelled nucleotides incorporated into the self-primed portion,
or any combination thereof.

24. (canceled)
